(19) United States
(12) Patent Application Publication
(10) Pub. No.: US 2016/0228387 A1
(43) Pub. Date: Aug. 11, 2016

(54) SOLUBILIZED FORMULATION OF COQ10 FOR USE IN TREATMENT OF PARKINSON'S DISEASE

(71) Applicants: NATIONAL RESEARCH COUNCIL OF CANADA (NRC), Ottawa (CA); UNIVERSITY OF WINDSOR, Windsor (CA)

(72) Inventors: Marianna SIKORSKA, Ottawa (CA); Jagdeep SANDHU, Ottawa (CA); Siyaram PANDEY, Lasalle (CA)

(73) Assignees: NATIONAL RESEARCH COUNCIL OF CANADA, Ottawa, ON (CA); UNIVERSITY OF WINDSOR, Windsor, ON (CA)

(21) Appl. No.: 15/028,520

(22) PCT Filed: Oct. 10, 2014
(86) PCT No.: PCT/CA2014/000735

§ 371 Date: Apr. 11, 2016

Related U.S. Application Data
(60) Provisional application No. 61/889,592, filed on Oct. 11, 2013.

(51) Int. Cl. A61K 31/122; A61K 9/00; A23L 2/52; A61K 31/353

(52) U.S. Cl. CPC A61K 31/122; A61K 31/353; A61K 9/0095; A61K 9/0053; A23L 1/30; A23L 2/52; A23L 1/296; A23V 2002/00

(57) ABSTRACT

Provided herein are compositions and methods for reducing neurogeneration in a patient suffering from Parkinson's Disease, comprising administration of a composition comprising CoQ10 and polyoxyethanyl-a-tocopherylsebacate (PTS). The compositions of the invention have been shown to penetrate the blood-brain barrier in mammals, thus effecting delivery of CoQ10 directly to brain tissue. Beneficial effects of the treatment have been observed at lower doses of CoQ10 than previously described.

### CROSS-REFERENCE TO RELATED APPLICATIONS

This application claims the benefit of U.S. Provisional Patent Application Ser. No. USSN 61/889,592 filed Oct. 11, 2013, the entire contents of which are hereby incorporated by reference

### FIELD OF THE INVENTION

The present invention relates to compositions for treatment of Parkinson's Disease and related methods.

### BACKGROUND OF THE INVENTION

Parkinson's disease (PD), the second most common neurodegenerative disorder, is characterised by the loss of dopaminergic (DA) neurons in the substantia nigra pars compacta (SNpc) region of the brain. PD affects approximately 1-2% of the population above the age of 55 and, in societies with an ageing population, disease management is a growing concern for neurologists and other physicians. By the time the characteristic features of PD such as bradykinesia, rigidity, postural instability, and resting tremor become obvious, approximately 60-70% of DA neurons in the SNpc have been lost. Currently, there is no therapy available to halt the progression of this neurodegeneration. It has been possible, however, to alleviate the symptoms of the disease by providing dopamine replacement by administration of levodopa. While levadopa is the most commonly utilized treatment for symptomatic relief, its prolonged application leads to drug-induced dyskinesia, which adversely affects the patients' quality of life.

In a majority of cases, the cause of PD remains unknown but factors contributing to the pathogenesis of the disease have been extensively studied. PD can be caused by environmental factors such as exposure to herbicides and pesticides or by genetic factors linked to gene mutations that increase susceptibility to PD. Although these genetic defects account for only 10% of PD cases, their identification brings about a better understanding of the disease pathophysiology and its progressive nature. It is known that classical symptoms of PD can be caused by exposure to the neurotoxin 1-methyl-4-phenyl-1,2,3,6-tetrahydropyridine (MPTP). It has been shown that MPTP injections cause selective loss of DA neurons in the SNpc region of certain strains of mice thereby creating animal models of PD. Although MPTP is not an environmental toxin and humans are not commonly exposed to it, several epidemiological studies reveal a link between the use of herbicides and pesticides such as paraquat (PQ), maneb and rotenone and the incidence of PD. The active metabolite of MPTP, MPP+, and PQ have structural similarity. They enter the DA neurons via the dopamine transporter and trigger neurodegeneration. Exposure to PQ has been shown to cause an increased susceptibility to PD. In rodents, PQ exposure leads to the loss of DA neurons in the SNpc region of the brain in a time and dose dependent manner. Therefore, rat and mouse models of PQ-induced neurodegeneration have been developed to study the pathophysiology of the disease and to develop successful treatment strategies.

One consistent finding between the PD patients and animal models of PD (MPTP, PQ, rotenone) is the malfunctioning of complex I of the electron transport chain, suggesting clearly that mitochondrial dysfunction is at the centre of PD pathophysiology. It appears that a blockage of complex I of the oxidative phosphorylation pathway by these toxins and the inability of DA neurons to cope with the excess of generated free radicals are the triggers of neuronal death. Therefore, it may be possible to interfere with the progression of neurodegenerative processes by applying antioxidants, which are capable of reducing the levels of free radicals. Numerous antioxidant compounds, some directly targeting mitochondria, have been investigated, but none have been used yet as an effective disease therapy.

One potential candidate for PD therapy is CoQ10 (2,3-dimethoxy, 5-methyl, 6-polyisoprene para-benzoquinone) because of its fundamental role in cellular energy production and antioxidant properties. CoQ10, also known as ubiquinone 50, is a lipophilic, redox active molecule located in all cellular membranes. CoQ10 has the formula shown below. The Q refers to the quinone head and the 10 refers to the number of isoprene units in the tail portion of the molecule.

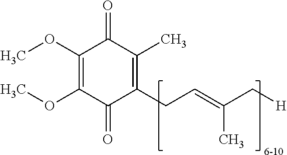

The 50 carbon polyisoprene chain of CoQ10 enables insertion in cellular membranes and the quinone ring, which undergoes reduction and/or oxidation transitions, becomes a carrier of protons and electrons. In the mitochondrial membrane, CoQ10 is an essential component of the mitochondrial respiratory chain where it transfers electrons from complex I and II to complex III and is an inhibitor of the mitochondrial permeability transition pore. CoQ10 undergoes oxidation and/or reduction in other cell membranes, such as Golgi vesicles, lysosomes, or plasma membrane, where it modulates vesicles acidification, subcellular redox state and is responsible for the generation of superoxide anion and hydrogen peroxide, which constitute a major regulatory signaling system essential for normal cell function and metabolism. In most membranes CoQ10 exists in the reduced form of quinol and acts as a powerful antioxidant protecting cells from reactive oxygen species (ROS) induced damage either by direct reaction with ROS or by regenerating α-tocopherol and ascorbate.

However, CoQ10 is a lipid soluble compound, characterized by limited bioavailability and it is difficult to deliver systemically, especially to the brain. Numerous early studies showed that CoQ10 was effective in preventing cell death caused by toxins such as PQ, however, very high doses of CoQ10 (from oil soluble formulations available on the market) were required to provide neuroprotection in vivo (Spindler et al., Neuropsychiatr Dis Treat 2009, 5:597-610). Oil soluble CoQ10 as a treatment for PD was tested in clinical trials in 2011, but phase 2 clinical trials were not successful. In pre-clinical work, the oil-soluble CoQ10 treatment was tested prophylactically using an MPTP PD-induced mouse model (Cleren C. et al, Neurochem. 2008, 104(6):1613-1621, Yang L. et al, J. Neurochem. 2009, 109(5):1427-1439). The oil soluble CoQ10 was shown to be effective for neuroprotection, but only at very large dosages (1,600 mg/kg/day). When this dosage is converted to a human dose (averaging 70 kg), it is112 g/day, which is beyond the acceptable FDA approved dose for clinical trials (2.4 g).

More recently, clinical studies in humans have shown that CoQ10 has no clinical benefit against PD, even when combined with Vitamin E to enhance uptake (Schapira et al, JAMA Neurol. 2014, 71(5):537-538 and 543-552). Even a version of CoQ10 which was chemically modified to efficiently cross cell membranes was shown to have no clinical benefit (Snow et al, Movement Disorders, 2010, 25(11):1670-4). Accordingly, in order for CoQ10 to be an effective treatment for PD, a need exists for improvements to solubility, absorption and brain penetration.

### SUMMARY OF THE INVENTION

The invention relates to a composition for use in treatment of Parkinson's Disease.

In one embodiment, the invention relates to a method for reducing neurogeneration in a patient suffering from Parkinson's Disease, said method comprising administration of a composition comprising CoQ10 and polyoxyethanyl-a-tocopherylsebacate (PTS).

In an aspect of the invention, the composition can be administered at low doses.

In another embodiment, the invention relates to a method for delivery of CoQ10 to brain tissue in a patient, said method comprising administration of Ubisol Q10 to the patient.

In another embodiment, the invention relates to a pharmaceutical composition comprising CoQ10 and polyoxyethanyl-a-tocopherylsebacate (PTS) for use in reducing neurogeneration in a patient suffering from Parkinson's Disease.

### BRIEF DESCRIPTION OF THE DRAWINGS

In order that the invention may be more clearly understood, embodiments thereof will now be described in detail by way of example, with reference to the accompanying drawings, in which:

FIG. 1 a) is a bar chart showing the level of CoQ10 in rat brain over a 6 hour period following administration of Ubisol-Q10 at a concentration of 50 μg/ml b) shows the same data wherein levels of CoQ10 in the brain are expressed as a percent of control;

FIG. 2 a) is a bar chart showing the total number of TH positive neurons in brain of rats sacrificed 0 weeks, 4 weeks and 8 weeks after PQ injections b) shows the same data wherein total number of TH positive neurons is expressed as a percent of control;

FIG. 3 is a bar chart showing the percentage decrease in TH positive neurons in brain of rats administered PQ alone and administered PQ+Ubisol-Q10;

FIG. 4 is a bar chart showing the percentage decrease in TH positive neurons in brain of rats administered PQ and then administered regular water for 8 weeks, Ubisol-Q10 for 8 weeks or Ubisol-Q10 for four weeks followed by regular water for 4 weeks; and

FIG. 5 is a bar chart showing the mean total number of leg slips made by rats administered PQ and then administered regular water for 8 weeks, Ubisol-Q10 for 8 weeks or Ubisol-Q10 for four weeks followed by regular water for 4 weeks.

### DETAILED DESCRIPTION OF PREFERRED EMBODIMENTS

Provided herein is a composition comprising CoQ10 which is capable of traversing the blood-brain barrier, thus enabling delivery of CoQ10 directly to the mammalian brain. The composition of the invention is useful in the treatment of PD.

The compositions of the invention consist of a nanomiscelle formulation of CoQ10 called Ubisol-Q10 which is water soluble. Ubisol-Q-10 contains CoQ10 and a derivatized form of α-tocopherol (vitamin E) called polyoxyethanyl-a-tocopherylsebacate (PTS) which has been shown to be an effective solubilizer. The structure and evaluation of PTS as a solubilizer was previously reported (Borowy-Borowski et al, J. Drug. Targ, 2004: 12(7): 415-424.).

The two components of Ubisol-Q10, CoQ10 and PTS, are combined at a ratio 1:2 mol/mol.

The general formula for PTS is:

X—OOO—(CH₂)n—COO—Y

where X is α-tocopherol and Y is polyethylene glycol (PEG). The PTS molecule is an amphiphile, possessing both hydrophilic (PEG) and lipophilic (α-tocopherol) properties, separated by an aliphatic spacer sebacic acid, and has self-emulsifying properties. Polyethylene glycols are commercially available under the trade name PEG, usually as mixtures of polymers characterized by an average molecular weight. Polyethylene glycols having an average molecular weight from about 300 to about 5000 are preferred, those having an average molecular weight from about 600 to about 1000 being particularly preferred.

PTS is part of a family of solubilizing agents, previously described in U.S. Pat. No. 6,045,826, having the formula:

{X—OOC—[(CH₂)ₙ—COO]ₘ}ₚ—Y

wherein:

X is a residue of a hydrophobic moiety,

Y is a residue of a hydrophilic moiety,

P is I or 2,

m is 0 or 1, and

n is an integer greater than or equal to 0.

The hydrophobic moiety of the solubilizing agent is a hydrophobic (lipophilic) molecule having an esterifable hydroxy group and is preferably a sterol or a tocopherol, in particular cholesterol, 7-dehydrocholesterol, campesterol, sitosterol, ergosterol, stigmasterol, or an a-, b-, g-, or d-tocopherol. Cholesterol and sitosterol are preferred sterols, sitosterol being particularly preferred. α-(+) Tocopherol and α-(±)-tocopherol are preferred tocopherols, α-(+)-tocopherol (vitamin E) being particularly preferred. Specific examples of solubilizing agents in this family are polyoxyethanyl-sitosterol sebacate (PSS), polyoxyethanyl-cholesteryl sebacate (PCS) and polyoxyethanyl-α-tocopheryl sebacate (PTS). This family of solubilizing agents show excellent solubility in water and allow the preparation of aqueous solutions of lipophilic compounds which shown excellent stability over time. However, surprisingly, compositions comprising PCS or PSS and CoQ10 were not effective to provide neuroprotection against induced PD in in culture models.

Based on the chemical structure, a-tocopherol constitutes 35.6% or one-third of the PTS molecule (Borowy-Borowski et al., 2004). When combined with CoQ10 and water, PTS facilitates the formation of nanomicelles. Based on transmission electron microscopy analyses, a single PTS-CoQ10 micelle measures 22±7 nm in diameter. CoQ10 added directly to water floats on the surface as insoluble material, whereas Ubisol-Q10 is fully dispersed in water and remains as a stable clear solution for up to 2 years or more, even at room temperature.

Previously, Ubisol-Q10 was tested in cell culture models and was shown to be efficient in protecting neurons from the toxic effects of PQ (Somayajulu M., Neurobiol. Dis. 2005, 18:618-625). It has also been tested in vivo in rats exposed to PQ (Somayajulu M., BMC Neurosci. 2009, 10:88) to determine whether prophylactic treatment would have a neuroprotective effect. However, PD is not diagnosed until symptoms arise, which occurs when almost 50-60% neurons are lost. Therefore, previous studies which demonstrated a prophylactic effect are not relevant to whether Ubisol Q10 is an effective treatment for PD, which requires a therapeutic treatment that can halt further neurodegeneration.

The compositions provided herein have been shown to effectively halt the neurodegeneration associated with PD. Furthermore, the compositions provided herein have beneficial effects at low daily dosages of CoQ10. In particular, the compositions of the invention are effective at CoQ10 daily doses of 30 mg/kg body weight (b.w.) or less, preferably 10 mg/kg b.w. or less and most preferably 6 mg/kg b.w. The beneficial effects were achieved at a much lower dose of CoQ10 compared to an oil soluble formulation, which was used at 200-1600 mg/kg/day in mice (Cleren C et al, 2008, 104(6):1613-1621). If the mouse dosage (6 mg/kg b.w) of the inventive composition was converted for human treatment, it would be 0.42 g/day, which is not only lower than the FDA approved amount for a clinical trial (2.4 g) but also lower than the approved maximum daily dosage for general supplement intake (1.2 g).

The inventors have now shown that a therapeutic administration of Ubisol-Q10 in rats already exposed to PQ halts the on-going neurodegeneration and behavioural deterioration associated with PD. As discussed further in the examples below, Ubisol-Q10 treatment saved close to 17% of neurons which would have otherwise died as a consequence of PQ exposure. This unprecedented neuroprotection has never been reported in animal models of neurotoxicity and offers a treatment to PD patients for better disease management. However, continuous Ubisol-Q10 supplementation was required to maintain the achieved level of neuroprotection.

The inventors have also used another model of PD, MPTP-induced PD, to demonstrate the effectiveness of Ubisol-Q10. The inventors have shown that orally administered Ubisol-Q10 in mice, in which MPTP had already initiated neurodegeneration, blocked the neuronal death pathway allowing the DA neurons to survive as long as the supplementation was continued (for at least 8 weeks post-MPTP treatment). However, when the supplementation was withdrawn, the neurodegeneration resumed and the neurons began to die. The neuroprotective Ubisol-Q10 treatment brought about a robust astrocytic response (activation) suggesting that these cells played a significant role in protecting the neurons.

The Ubisol-Q10 formulation of CoQ10 is FDA-GRAS approved and preliminary toxicity results show that there is no overt toxicity even when the dose is increased to 10 times the dose required to halt neurodegeneration in PD.

There are multiple explanations which could explain how Ubisol-Q10 protects the remaining neurons following the onset of neurodegeneration. Until recently, one theory was that the combined antioxidant nature of the two components of Ubisol-Q10 (CoQ10 and Vitamin E) could quench the levels of oxidative stress associated with the disease. Previously, it has been shown that vitamin E alone did not have a significant effect on neuroprotection (Somayajulu M, BMC Neurosci 2009, 10:88). However, as discussed above, a large recent study has shown that the combination of CoQ10 and Vitamin E had no benefit in treating PD (Schapira et al, JAMA Neurol. 2014; 71(5):537-538 and 543-552).

Another possible explanation for the effectiveness of Ubisol-Q10 is that the water soluble composition makes absorption into the blood stream easier, therefore, making it possible for the CoQ10 to cross the blood brain barrier. Previous reports have shown elevated plasma content of CoQ10 in rodents and humans following injection. However, the inventors are not aware of any previous reports of CoQ10 penetrating the blood-brain barrier. The inventors have, surprisingly, demonstrated that Ubisol-Q10 crosses the blood-brain barrier and delivers CoQ10 directly to brain tissue within one hour of administration. Following administration of Ubisol-Q10, there is an increase in brain content of CoQ10 of 35% after 3 hours. Another significant finding was that, once in the brain, CoQ10 does not accumulate. This means that there is no build-up of CoQ10 in the brain, which could be toxic to the neurons. The natural removal explains why withdrawal of the treatment terminates the therapeutic effect. Thus, in order to sustain neuroprotection, the treatment must be continuous and, in doing so, neurotoxicity will not result. Therefore, it appears that Ubisol-Q10 does not halt neurodegeneration by acting on the toxin, but rather by supporting the remaining neurons.

Another surprising effect of Ubisol-Q10 treatment was that the neuroprotective effect of the composition was rapid. Specifically, the neuronal death pathway was blocked after the Ubisol-Q10 treatment was commenced. This is an important and unexpected advantage of Ubisol-Q10 treatment because PD is a degenerative condition; t any delay in the effect of treatment results in continued neuron death and deterioration in patient health. A treatment that immediately halts further degeneration associated with PD is of significant benefit to patients.

Compositions of the invention can be administered orally in liquid form, as a medicament or as an additive to beverages.

### EXAMPLE 1

### Bioavailability Analysis—Levels of CoQ10 in Rat Brain

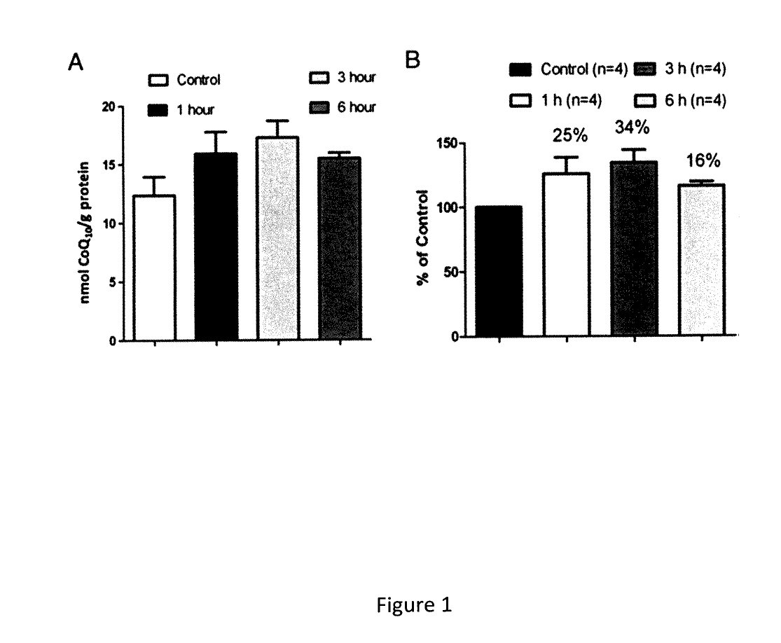

The brain CoQ10 levels were measured in rats which were given a 1 h access to Ubisol-Q10 supplemented water (at a concentration of 50 μg/ml) after a 24 h period of water deprivation. During this time, rats drank on average 10 ml of solution containing 500 μg of CoQ10. Animals were sacrificed at different time points after the Ubisol-Q10 intake. Brain tissue was collected and CoQ10 content was measured as previously described [Graves S, et al. Methods Mol Biol 1998, 108:353-365]. Briefly, samples were homogenized in cold PBS and subjected to repeated freezing/thawing steps to disrupt protein/lipid complexes. CoQ10 was extracted and analysed by HPLC following separation on a TSK-GEL ODS-100S column (4.6 mm×150 mm, 7μ particle size, TOSOH Biosep LLC, Montgomeryville), equipped with a 1 mm C18 guard column (Optimize Technologies Inc., Oregon City, Oreg.). Absorbance at 275 nm was monitored and recorded using Beckman System Gold Software CoQ10 was extracted and analysed by HPLC. The results are shown in FIG. 1 and in Table 1 below. A modest, but time dependent elevation of CoQ10 in the brain was evident within 1 hour post-gavage (3-fold over the basal level). It peaked at 3 hours (5-fold over the basal level), and remained elevated for up to 24 hours.

TABLE 1

| Control (n = 4) | 1 h (n = 4) | 3 h (n = 4) | 6 h (n = 4) |
|---|---|---|---|
| 13.6 ± 2.7 | 17.21 ± 3.6 | 18.3 ± 2.7 | 15.8 ± 0.92 |

Data is shown as mean ± SD

The other component of Ubisol-Q10 formulation, α-tocopherol (vitamin E) was systemically released as revealed by HPLC analyses. Thus, pharmacokinetic distribution of the two Ubisol-Q10 components followed separate paths, especially because the release of α-tocopherol required the hydrolysis of PTS to its primary components. The measured molar concentration of CoQ10 in control brains was 4-fold higher than vitamin E (approximately 154 pmol/mg protein vs. 43 pmol/mg protein) and a similar ratio of the 2 antioxidants seemed to be maintained following Ubisol-Q10 intake as the maximal tissue increases for both compounds ranged between 4- and 5-fold. Similar to the brain levels, plasma levels of both CoQ10 and vitamin E were also elevated within 1 hour.

### EXAMPLE 2

### Treatment of Rats with PD Neurodegeneration Induced by PQ Using Ubisol-CoQ10

Long Evans Hooded rats were given 5 intraperitoneal injections of PQ at a dose of 10 mg/kg body weight/injection dissolved in phosphate buffered saline (PBS), one injection every five days over a period of 20 days. Control rats received intraperitoneal injections of PBS alone. Brain tissue was examined immediately after the last PQ injection and, subsequently, 4 weeks and 8 weeks later. Supplementation of drinking water with Ubisol-Q10 at a concentration of 200 μg/ml (equivalent to 50 μg CoQ10/ml) began on the day of the last PQ injection and it was continued for either 4 weeks or 8 weeks. Fresh drinking solutions were provided every second day. At the conclusion of experimental treatments, rats were perfused with Tyrodes buffer containing heparin, the tissues were fixed with 10% formalin, and the brains extracted and stored in the 10% formalin until processing for immunohistochemistry. Animals were sacrificed at different time points for up to 8 weeks post-PQ exposure. Midbrain sections were prepared, immunostained with anti-tyrosine hydroxylase antibody and TH-positive neurons were counted using a stereologer in an unbiased manner.

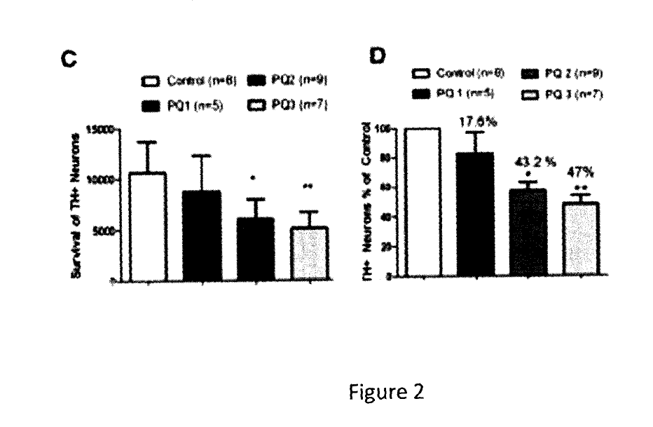

A substantial percentage of DA neurons, i.e. close to 18%, were lost during the PQ injection period (PQ1 group). The neurons continued to die over the next several weeks reducing the number of TH-positive neurons by 43% at the end of week 4 (PQ2 group) and by 47% at the end of week 8 (PQ3 group) post-PQ exposure (See FIG. 2 and Table 2 below). This model is probably closest to what happens in humans as one month in a rat's lifetime is equivalent to 2.5 years in human.

TABLE 2

| Control (n = 8) | PQ1 (n = 5) | PQ2 (n = 9) | PQ3 (n = 7) |
|---|---|---|---|
| 10585 ± 3169 | 8722 ± 3559 | 6006 ± 1928 | 5114 ± 1562 |

Data is shown as mean ± SD

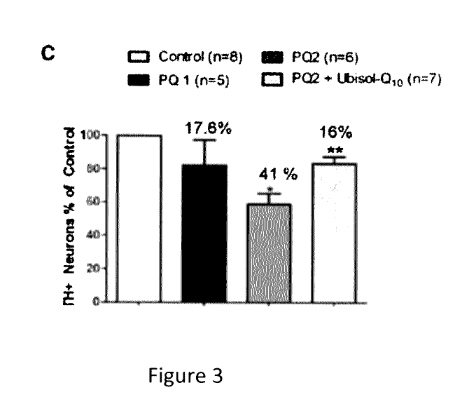

The Ubisol-Q10 intervention was applied after the completion of PQ injections described above. By this time, neurodegenerative processes in the brain had already begun. The PQ-treated group of rats was placed on Ubisol-Q10 supplemented drinking water (containing 50 μg/ml of CoQ10) for 4 weeks (PQ2+ 4 wks Ubisol-Q10 group). This treatment began when nearly 18% of SN neurons were already lost (PQ1 group), but the question was whether the remaining vulnerable neurons could be saved. The generated data is summarized in FIG. 3 and Table 3, below. The midbrain sections were immunostained with anti-TH antibodies, and the stained neurons were counted using a stereologer in an unbiased manner. As discussed above (FIG. 2), the PQ-treated rats drinking regular water (PQ2 group) lost over 40% of DA neurons over the period of 4 weeks, whereas rats drinking Ubisol-Q10 lost less than 20% (PQ2+ 4 wks Ubisol-Q10 group). Ubisol-Q10 treatment saved close to 17% of neurons which would have otherwise died as a consequence of PQ exposure.

TABLE 3

| | | | PQ2 + Ubisol-Q10 |
|---|---|---|---|
| Control (n = 8) | PQ1 (n = 5) | PQ2 (n = 6) | (n = 7) |
| 10585 ± 3169 | 8722 ± 3559 | 6227 ± 1682 | 8845 ± 1088 |

Data is shown as mean ± SD

### EXAMPLE 3

### Ubisol-Q10 Supplementation Required to Maintain Neuroprotection

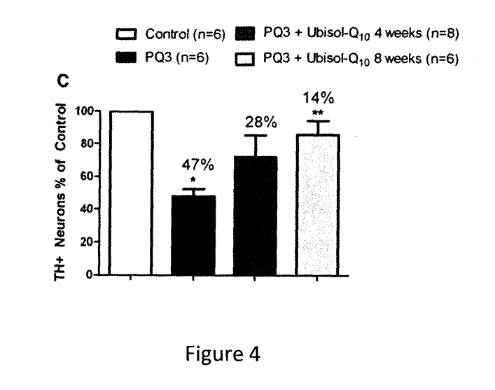

PQ treated rats were either given regular drinking water for 8 weeks post PQ, kept on Ubisol-Q10 for 8 weeks post-PQ or given Ubisol-Q10 for 4 weeks and then regular tap water for the additional 4 weeks (8 weeks total). There was a significant loss of DA neurons in the rats given PQ but no Ubisol-Q10, approximately 47% in comparison with the saline injected control group indicating progressive neurodegeneration over a period of eight weeks. There was also significant neuroprotection in the rats that received the Ubisol-Q10 supplemented drinking water for eight weeks post injections. The Ubisol-Q10 intervention began after nearly 15% of DA neurons were already killed, no further loss of neurons was observed in this group (FIG. 4 and Table 4, below) which show that only 14% of neuronal loss was recorded. However, if the treatment was withheld after 4 weeks, the neurodegeneration resumed as evidenced by the reduced number of surviving neurons in this experimental group comparing to the group receiving Ubisol-Q10 for 8 weeks (28% versus 14%, respectively). Therefore,continuous Ubisol-Q10. supplementation was required to maintain the achieved level of neuroprotection.

TABLE 4

| | | PQ3 + Ubisol-Q10 | PQ3 + Ubisol-Q10 |
|---|---|---|---|
| Control (n = 6) | PQ3 (n = 6) | 4 weeks (n = 8) | 8 weeks (n = 6) |
| 9717 ± 3321 | 4661 ± 1097 | 6397 ± 3648 | 8366 ± 2053 |

Data is shown as mean ± SD

### EXAMPLE 4

### Effect of Ubisol-Q10 on Motor Function Deterioration

Deficiency in motor function is a hallmark of PD. Motor skills of the rats treated in Example 2 were assessed using the beam walk test. All rats were assessed for performance on a horizontal beam-walking test for motor skills/motor deficits as measured by leg slips. The aluminium beam was 1.68 metres in length, 2 centimetres in width and 0.75 metres from the ground. A mirror was placed behind the beam, measuring 1.78 metres in length and 0.3 metres in height. Four weeks after the last injection, rats underwent one trial per day for four consecutive days (one training trial and three test trials). Eight weeks after the last injection another three test trials were performed (one trial per day). In the training trial, rats ran down the beam to the holding cage on a flat platform three times, each time with different distances between the holding cage and starting position. The first position was a quarter of the beam length, the second was half, and the last was the entire distance of the beam. This last distance is where mice were placed for the subsequent test trials.

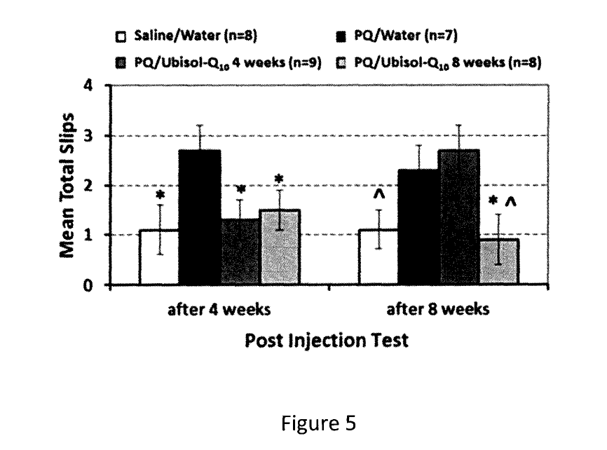

Rats received a small slice of apple in the holding cage located on a table at the end of the beam. The rat had up to 2 minutes to cross the beam. The test trials were recorded using a standard video camera, located 2 metres perpendicular to the beam. The number of hind leg slips made from either leg during each test trial was later noted from viewing the recorded video clips. The number of limb slips for each rat was summed over the three test sessions in each phase because rats made too few slips in each session to analyse this behaviour over trials within each session. The statistical analysis of each rat's total number of leg slips over each test series was carried by a two-way ANOVA (Groups x Test phase with repeated measures on the second factor). Effects from these analyses were considered significant at p<0.05. Post-hoc comparisons between groups at each test phase were carried out by Least Squares Difference post-hoc multiple comparisons test multiple comparisons and significant differences between groups were considered at p<0.05 (one-tail) based on the prediction that rats not given post-injection Ubisol-Q10 in their drinking water would show deficits associated with PQ-induced neurodegeneration. Results are shown in FIG. 5.

The PQ3 group made more leg slips than either the control or the PQ3+ Ubisol-Q10 8 weeks in both the test phases or than the PQ3+ Ubisol-Q10 4 weeks group in the first test phase. The PQ3+ Ubisol-Q10 4 weeks increased its leg slips to the elevated levels of the PQ3 group in the second test phase. Multiple comparisons between groups confirmed that the number of leg slips of the PQ3 group was significantly greater than those of the other three groups (p<0.05) in the 1st test phase but only remained significantly greater than that of the PQ3+/Ubisol-Q10 8 weeks group in 2nd test phase (p=0.036). Thus, even though the observed groups by phase interact was not significant, multiple comparison between groups reveal that only those rats that received Ubisol-Q10 in their drinking water over the complete post injection period maintained their superior performance similar to that of rats that were not exposed to potential neurodegenerative effects of PQ. From a behavioral aspect, treatment with the neuroprotectant agent only half way through the post-injection period was not sufficient to maintain its effect to the end of the experiment.

### EXAMPLE 5

### Toxicity Study

Rats were maintained on drinking water supplemented with Ubisol-Q10 at a dose 10 times higher (60 mg/kg/day) than that used for neuroprotection (6 mg/kg/day) for 2.5 months. Animals were weighed once a week to ensure their health. The rats were then perfused with heparin containing Tyrodes buffer and formalin fixed tissue—heart, lung, liver and kidney were sent to the Animal Health Laboratory, University of Guelph. Hematoxylin & Eosin-stained histological sections of the tissues were evaluated by a board-certified veterinary pathologist. No overt lesions of toxicological significance were observed in the Ubisol-Q10 treated animals

During the dosing period, the Ubisol-Q10 treated rats never displayed any signs of discomfort, no change in eating, drinking, grooming habits and no difference in body weight in comparison with rats drinking regular tap water over the same time period.

### EXAMPLE 6

### Prophylactic Treatment of Mice with PD Neurodegeneration Induced by MPTP Using Ubisol-CoQ10

To induce the dopaminergic neurodegeneration, male C57BL/6 mice were acclimatized to the new environment for 7 days before the start of the experiment. Animals were randomly divided into experimental groups and given 5 daily injections of MPTP (25 mg/kg/injection). Control mice were injected with saline. On days 5, 8, 14, 28, and 45 after the MPTP injections, mice were sacrificed and brain tissue was collected for immunohistochemistry, stereology, and biochemical analyses. Brains were fixed and immunostained with rabbit polyclonal anti-tyrosine hydroxylase antibody (brown) and counterstained with cresyl violet (blue) for anatomic reference. TH-positive cells were counted using an unbiased stereology method and cell survival was plotted as percentage of control. The images of immunostained midbrain sections examined at day 28 (MPTP-D28) of the experiment revealed a significantly reduced TH-immunostaining and decreased number of SNpc neurons in the MPTP-injected mice in comparison to control animals. Similarly, the density of TH-positive fibers in striatum at the same time point (MPTP-D28) was much lower in the MPTP-treated group than in the controls. Accordingly, the TH-positive cell counts revealed that the neurons were progressively dying over a period of the first 28 days of the experiment, with overall cell loss of 51.6% at day 28. No further cell loss was observed during the subsequent days of the experiment as the same neuronal counts (i.e., 50% survival) were found on day 45 (MPTP-D45). The striatal dopamine content also decreased by close to 50% in MPTP-treated mice during that time period indicative of extensive degeneration of the nigrostriatal pathway. Thus, in the experimental paradigm used here, that is, 5-intraperitoneal injections of MPTP, the dopaminergic degeneration occurred over a period of 28 days, with approximately 25% of TH-positive neurons being killed after 5 days of MPTP injections (MPTP-D5), and a further 25% between the days 5-28 of the experiment, resulting also in the reduction of striatal dopamine by 50% (MPTP-D28). These markers of neurodegeneration plateaued at day 28 as neither the number of TH-positive neurons nor dopamine level declined further at day 45.

The effects of prophylactic supplementation of Ubisol-Q10 at 30 mg CoQ10/kg/day were investigated as follows. Mice were acclimatized for 7 days before the start of the experiment and were randomly divided into 3 experimental groups (I-III). Groups I and II were given regular water throughout the duration of the experiment. Ubisol-Q10 supplementation of drinking water (30 mg CoQ10/kg/day) in group III began 2 weeks before the MPTP injections (D[-14]) and was maintained until the termination of the experiment on day 28 (D28). Groups II and III received 5 daily intraperitoneal injections of MPTP (25 mg/kg/injection) whereas, group I was injected with saline. At the conclusion of the experiment on D28, all animals were sacrificed and brain tissue was collected for immunohistochemistry, sterology, and Western blot. The microscopic examination of midbrain sections revealed a clear reduction in the number of TH stained DA neurons in the MPTP-injected mice (group II) in comparison to saline-injected controls (group I). This observation was confirmed by cell counts, consistently showing 50% loss of TH neurons in SNpc at day 28 (compare groups I and II, p<0.001). By contrast, the TH-stained midbrain sections of MPTP-treated mice receiving the prophylactic supplementation of Ubisol-Q10 (group III) were significantly different from those without the supplementation (group II). The density of TH-positive neurons in the SNpc and DA fibers in striatum were nearly indistinguishable from those seen in the saline-injected control mice of group I. The cell counting established a greater than 80% survival of TH-positive neurons at D28 in this group of mice (p<0.01). Western blot analysis further confirmed the immunostaining and counting data, showing much stronger TH-immunoreactive band in the brain of MPTP mice receiving prophylactic Ubisol-Q10 rather than regular water. Therefore, the prophylactic application of Ubisol-Q10 at a dose of 30 mg CoQ10/kg b.w. effectively protected the mouse dopaminergic pathway against MPTP-induced neurodegeneration.

### EXAMPLE 7

### Action of Ubisol-Q10 Against MPTP

The results of the previously mentioned study posed a question whether the bioactive components of Ubisol-Q10, CoQ10, and vitamin E acted to neutralize MPP+ and prevented it from penetrating the brain. To answer this question, the MPP+ levels in the brain and liver samples were compared between MPTP-treated mice drinking regular water and water supplemented with Ubisol-Q10. The Ubisol-Q10 supplementation at 30 mg CoQ10/kg b.w. started 2 weeks before the injection but the mice received only a single intraperitoneal MPTP injection (25 mg/kg BW) and the samples were collected 90 minutes and 4 hours after the injection. Using HPLC based analysis of MPP+, the results revealed the presence of the neurotoxin in both liver and brain samples at the analyzed time points. For both tissues, the higher content of MPP+ was detected at 90 minutes than at 4 hours post-MPTP injection. Importantly, no statistically significant differences in the MPP+ levels between groups drinking regular water and Ubisol-Q10 supplemented water were identified showing clearly that Ubisol-Q10 supplementation did not interfere with the MPTP metabolism and the generation of the neurotoxic MPP+.

### EXAMPLE 8

### Treatment of Mice with PD Neurodegeneration Induced by MPTP with Ubisol-CoQ10

Neuroprotection by delaying the Ubisol-Q10 supplementation past the MPTP injections was investigated as follows. Mice were acclimatized to the new environment for 7 days before the start of the experiment and were randomly divided into 3 experimental groups (I-III). Control group I was injected with saline and groups II and III received 5 daily MPTP injections (25 mg/kg/injection). Mice in groups I and II were given regular drinking water throughout the duration of the experiment whereas mice in group III were placed on Ubisol-Q10 supplemented water starting on day 5 (D5) at 30 mg CoQ10/kg b.w./day, immediately after the last MPTP injection. The supplementation continued until the conclusion of the experiment on D28. Thus, the Ubisol-Q10 intervention begun when the neurodegeneration was already well under way as the trigger of neuronal killing, MPP+, was reaching the brain within 90 minutes of the MPTP injection and, by D5 of the experiment, over 25% of neurons were already lost.

At the conclusion of the experiment on day 28 (D28), all mice were sacrificed and brains were collected for immunohistochemistry and stereology. Brains were fixed and immunostained with rabbit polyclonal anti-tyrosine hydroxylase antibody (brown) and counterstained with cresyl violet (blue) for anatomic reference. Images were captured on an Olympus microscope equipped with Microcast 3CCD 1080p HD color camera system.

Consistent with the earlier experiments, microscopic examination of midbrain sections showed a similar reduction in TH immunostaining of both the cell bodies and the fibers in the MPTP-injected animals of group II in comparison to saline-injected control animals of group I. In striking contrast, the midbrain sections from MPTP treated mice receiving Ubisol-Q10 from D5 onward (group III) showed very well preserved TH-immunostained cell bodies as well as fibers and could hardly be distinguished from the control group I. These observations were also corroborated by the counts of surviving neurons (see Table 5 below). The number of TH neurons dropped by nearly 50% in the MPTP-treated animals of group II, however, a much higher number of surviving TH neurons (approximately 70%) were found in mice of group III receiving Ubisol-Q10. The Ubisol-Q10 treatment was initiated on D5 with approximately 75% of alive neurons and it culminated at D28 with nearly the same percentage of viable cells, that is 70%±6.4%, indicating that, once the Ubisol-Q10 intervention began, the neuronal death pathway was blocked.

TABLE 5

| | | MPTP + Ubisol-Q10 |
|---|---|---|
| Control (n = 16) | MPTP (n = 16) | (n = 14) |
| 100 ± 5.7 | 50.3 ± 7.3 | 69.6 ± 6.4 |

### EXAMPLE 9

### Treatment of Mice with Using Lower Dose Ubisol-CoQ10

Ubisol-Q10 doses of 6 mg/kg/day and 3 mg/kg/day were tested. Mice were acclimatized for 7 days before the start of the experiment and were randomly divided into 4 experimental groups (I-IV). Control group I was injected with saline and groups II-IV with MPTP (5×25 mg/kg). Mice in groups I and II were drinking regular water throughout the duration of the experiment. Mice in groups III and IV received Ubisol-Q10 supplemented water at 3 mg and 6 mg CoQ10/kg/day, respectively, starting immediately after the last injection (D5). At the conclusion of the experiment (D28), all mice were sacrificed and brains were collected for immunohistochemistry, stereology, and biochemistry. Brains were fixed and immunostained with rabbit polyclonal anti-tyrosine hydroxylase antibody and counterstained with cresyl violet. Images were captured on an Olympus microscope equipped with Microcast 3CCD 1080p HD color camera. Visual examination of midbrain sections showed a dramatic reduction of neurons in the SNpc of the MPTP-treated animals without supplementation (group II) and saving of the neurons in the MPTP-treated mice receiving the Ubisol-Q10 supplementation (group III). Higher magnification photomicrographs of SNpc from MPTP-treated animals (group II) revealed reduced TH-staining and pyknotic cells with shortened neuronal processes, displaying distinct neuritic beading as compared with the control group I. Significantly, such pathology was not evident in the Ubisol-Q10 treated mice of groups III and IV. Instead, a greater number of TH stained cells with round soma and well-preserved neuronal processes were seen. This was true for both Ubisol-Q10 concentrations tested, that is, 6 mg CoQ10/kg/day and 3 mg CoQ10/kg/day. Consistent with these observations, a quantification of TH-positive neurons, using a computerized stereologer system, showed much higher numbers of surviving dopamine neurons in the MPTP-Ubisol-Q10 treated groups III and IV than in MPTP alone of group II. Approximately 20% more TH-positive neurons in SNpc were found in groups III and IV compared with group II (p<0.05) (See Table 6 below).

TABLE 6

| | | | MPTP + |
|---|---|---|---|
| | | | Ubisol-Q10 |
| Dosage | Control | MPTP (n = 10) | (n = 10) |
| 6 mg/kg/day (n = 10) | 5528 ± 213 | 3281 ± 291 | 4709 ± 432 |
| 3 mg/kg/day (n = 6) | 6172 ± 529 | 3320 ± 700 | 5934 ± 660 |

As shown previously, the MPTP treatment (group II) reduced striatal dopamine level to 50% by D28. The drop in dopamine level was less severe in mice given the Ubisol-Q10 supplementation (group III), consistent with higher number of surviving TH-positive neurons in these animals (See Table 7, below).

TABLE 7

| | | MPTP + Ubisol-Q10 |
|---|---|---|
| Control (n = 6) | MPTP (n = 6) | (n = 9) |
| 38.04 ± 1.8 | 15.1 ± 2.2 | 18.2 ± 0.91 |

Taking into consideration the fact that the Ubisol-Q10 treatment began in the midst of ongoing neurodegeneration, and after 20%-25% of neurons were already gone, the therapeutic supplementation of Ubisol-Q10 offered neuroprotection against MPTP-induced neuronal killing; it stopped completely its further progression. The formulation was equally effective at a CoQ10 dose of 3 mg/kg/day as it was at a dose 10 times higher of 30 mg/kg/day, meaning that a 70 kg patient would require only 210 mg/day. At the conclusion of the experiment on D28, brain (cortex) CoQ10 content was measured to establish whether the 3 week Ubisol-Q10 supplementation altered its brain levels. There was no statistically significant difference in CoQ10 content between mice receiving Ubisol-Q10 supplementation (group III) and mice drinking regular water (groups I and II), indicating that the delivered CoQ10 must have been used in processes supporting the neuroprotection and did not accumulate in the brain. This was in contrast to transient elevation of brain CoQ10 in intact mice following a single bolus dose of Ubisol-Q10. No change in the content of α-tocopherol was seen at day 28.

As an additional step, the beam walk test, described above, was used to assess motor skills. The handling and training of mice on the test apparatus were performed before the MPTP injections. Mice were acclimatized to the new environment for 7 days (D[-21]-D[-14]) before the handling (D[-14]-D[-7]) and training (D[-7]-D1). Animals were randomly divided into 3 experimental groups (I-III). Group I (control) was injected with saline and groups II and III received 5 daily MPTP injections (25 mg/kg/injection; D1- is a bar chart showing the percentage decrease in TH positive neurons in brain of rats administered PQ and then administered regular water for 8 weeks, Ubisol-Q10 for 8 weeks or Ubisol-Q10 for four weeks followed by regular water for 4 weeks D5). Mice in groups I and II were given regular drinking water throughout the duration of the experiment whereas mice in group III were placed on Ubisol-Q10 supplemented water (6 mg CoQ10/kg/day) starting on day 5 immediately after the last MPTP injection (D5). Mice were tested on D10 (5 days on Ubisol-Q10), D17 (12 days on Ubisol-Q10), and on D24 (after 19 days on Ubisol-Q10). Animals were allowed to cross a 5-mm square and a 100-cm long beam and the number of faults, as well as the time taken to walk the beam, were recorded. Significantly fewer faults were recorded in mice receiving Ubisol-Q10 for only 5 days (group III), less than 4 faults on average in group III as compared with nearly 7 in group II (p<0.05), providing further evidence for the neuroprotective effects of Ubisol-Q10 supplementation. However, in the subsequent tests on D17 and D24, the differences between the groups were less obvious. Although the same trend toward the improvement of motor skills in the Ubisol-Q10 treated mice was seen; the data did not reach statistical significance. This was consistent with the abilities of mice to adapt and learn.

### EXAMPLE 10

### Period of Ubisol-Q10 Supplementation Required to Maintain Neuroprotection Following MPTP Treatment

Mice were acclimatized to the new environment and were randomly divided into 4 experimental groups (I, II, V, and VI). Group I was injected with saline and groups II, V, and VI were given 5-daily (D1-D5) injections of MPTP (25 mg/kg/injection). Mice of groups I and II were drinking regular water until the termination of the experiment on day 56 (D56). Mice in group V were given Ubisol-Q10 supplemented water (6 mg CoQ10/kg/day) from D5 till D28 (3 weeks), and then they were switched to regular water for the rest of the experimental period (till D56 or for additional 4 weeks). Mice in group VI were placed on the same Ubisol-Q10 supplementation from D5 till D56 (total 7 weeks). At the conclusion of the experiment on D56, mice were sacrificed and brain tissue was collected for immunohistochemistry and stereology. Brains were fixed and immunostained with rabbit polyclonal anti-tyrosine hydroxylase antibody (brown) and counterstained with cresyl violet (blue). Images were captured on an Olympus microscope equipped with Microcast 3CCD 1080p HD color camera. The outcomes were assessed based on TH immunochemistry and stereological counting of TH positive neurons in the SNpc region. The data is shown in Table 8, below. The neuroprotection delivered by the 7 week Ubisol-Q10 supplementation (group VI) was similar to uninterrupted 3 weeks supplementation in group III in the experiment. Furthermore, the number of surviving neurons after 7 weeks of treatment (group VI) was nearly the same as it was at the start of the treatment on D5 suggesting, that this treatment continued to block the progression of neurodegeneration. However, the data also revealed that if the supplementation was withdrawn after 3 weeks and the animals were given regular drinking water for the subsequent 4 week period (group V), the neurodegeneration resumed. This resulted in fewer viable neurons being accounted for in group V at the termination of the experiment. These results showed that the bioactive components of Ubisol-Q10 were capable of penetrating and blocking the molecular pathway(s) activated by MPTP and responsible for the death of DA neurons, but their ongoing supply was needed to maintain that block.

TABLE 8

| | | MPTP + | MPTP + |
|---|---|---|---|
| Control | | Ubisol-Q10_3 wk | Ubisol-Q10_7 wk |
| (n = 10) | MPTP (n = 8) | (n = 14) | (n = 12) |
| 6336 ± 273 | 4192 ± 295 | 5008 ± 299 | 5794 ± 264 |

### EXAMPLE 11

### Use of CoQ10 with PCS Solubilizer in Mouse Model

Male C57BL/6 mice, 8-10 wk old (20-25 g) were divided into five groups. Groups I-IV received the following formulations in drinking water starting 2 weeks before MPTP injections and then for 3 more weeks after the MPTP injections. Control animals received saline injections only. All other mice were received 5 intraperitoneal injections of MPTP-HCl (25 mg/kg body weight/injection; Sigma Aldrich), once a day for 5 days. Group 1 received PTS in drinking water, Group II received UbisolQ10, Group III received CoQ10/PCS and Group IV received PCS alone. At the termination of the experiment, mice were anesthetized with isofluorane and perfused transcardially with 10 mL ice-cold 1× phosphate-buffered saline (PBS) followed by 10% neutral buffered formalin (Fischer Scientific) and embedded in paraffin. Brains were cut throughout the substantia nigra and every 5^{th} section was processed for tyrosine hydroxylase immunohistochemistry. The number of tyrosine hydroxylase-positive neurons were counted. The results are shown in FIG. 6 and in Table 9, below. As is shown, CoQ10/PCS showed no neuroprotective affect.

TABLE 9

| | Control | MPTP | CoQ10/PCS | PCS |
|---|---|---|---|---|
| Mean | 100 | 66 | 67 | 58 |
| Std Deviation | 21 | 23.4 | 19.3 | 28.5 |

### EXAMPLE 12

### Further Bioavailability Analysis—Levels of CoQ10 and Vitamin E in Mouse Brain

Mice were given Ubisol-Q10 (6 mgCoQ10/kg BW) by gavage and were sacrificed 1, 3, 6 and 24 hours later. CoQ10 and vitamin E were extracted from the brains and analyzed by HPLC. Brain contents of CoQ10 following Ubisol-Q10 ingestion showed statistically significant differences between control versus 1, 3, 6, and 24 hours groups as shown in Tables 10 and 11 below. Data is shown in pmoles/mg of brain tissue as mean±SD.

TABLE 10

| Control | | | | |
|---|---|---|---|---|
| (n = 10) | 1 h (n = 10) | 3 h (n = 5) | 6 h (n = 5) | 24 h (n = 5) |
| 153.6 ± 7.3 | 444.5 ± 67.5 | 620 ± 73.4 | 445.6 ± 64.7 | 378 ± 24 |

TABLE 11

| Control (n = 6) | 1 h (n = 8) | 3 h (n = 8) | 6 h (n = 8) |
|---|---|---|---|
| 42.5 ± 3.8 | 171.5 ± 23.15 | 95.71 ± 13.8 | 111.3 ± 14.6 |

Although the foregoing invention has been described in some detail by way of illustration and example for purposes of clarity, it would be apparent to those skilled in the art that certain changes and modifications are within the scope of the invention. Therefore, the description and examples should not be construed as limiting the scope of the invention as claimed.

1. A method for reducing neurogeneration in a patient suffering from Parkinson's Disease, said method comprising administration of a composition comprising CoQ10 and polyoxyethanyl-a-tocopherylsebacate (PTS).

2. The method of claim 1 wherein the composition is administered in a dose of 200 to 4000 milligrams per day.

3. The method of claim 1 wherein the composition is administered in a dose of 200 to 1000 milligrams per day.

4. The method of claim 1 wherein the composition is administered in a dose of 200 to 500 milligrams per day.

5. The method of claim 1 wherein the composition is administered orally.

6. A method for delivery of CoQ10 to brain tissue in a patient, said method comprising administration of Ubisol Q10 to the patient.

7. The method of claim 6 wherein the composition is administered in a dose of 200 to 4000 milligrams per day.

8. The method of claim 6 wherein the composition is administered in a dose of 200 to 1000 milligrams per day.

9. The method of claim 6 wherein the composition is administered in a dose of 200 to 500 milligrams per day.

10. The method of claim 6 wherein the composition is administered orally.

11. A pharmaceutical composition comprising CoQ10 and polyoxyethanyl-a-tocopherylsebacate (PTS) for use in reducing neurogeneration in a patient suffering from Parkinson's Disease.

12. The pharmaceutical composition of claim 5 wherein the CoQ10 and PTS are present a ratio 1:2 mol/mol.

13. The pharmaceutical composition of claim 5 which is a composition for oral administration.

14. A nutritional supplement comprising CoQ10 and polyoxyethanyl-a-tocopherylsebacate (PTS) for use in reducing neurogeneration in a patient suffering from Parkinson's Disease.

15. The nutritional supplement of claim 14 wherein the CoQ10 and PTS are present a ratio 1:2 mol/mol.

16. The nutritional supplement of claim 14 which is intended for use in a beverage.
